(19) United States
(12) Patent Application Publication
(10) Pub. No.: US 2012/0089170 A1
(43) Pub. Date: Apr. 12, 2012

(54) INTRAGASTRIC BALLOON GEOMETRIES

(75) Inventor: Zachary Dominguez, Santa Barbara, CA (US)

(73) Assignee: ALLERGAN, INC., Irvine, CA (US)

(21) Appl. No.: 12/902,085

(22) Filed: Oct. 11, 2010

(51) Int. Cl. A61M 29/02; A61M 29/00

(52) U.S. Cl. 606/192; 606/191

(57) ABSTRACT

The present application provides intragastric devices for the treatment of obesity. The intragastric devices advantageously act as a volume-occupying device, and is able to survive implantation in a patient's stomach for a year or longer. In addition, the intragastric devices may provide additional benefits. For example, the intragastric device may be configured to stimulate an inner stomach wall and/or temporarily block the pylorus to slow gastric emptying and/or be rotationally variant, thereby encouraging different stimulation points on the inner wall of the stomach and limiting the stomach's ability to adapt over long term implantation.

### FIELD

The present application relates, in general, to devices and methods for controlling obesity, and, more particularly, to an intragastric device designed to promote satiety by occupying volume in a patient's stomach.

### BACKGROUND

Severe obesity is an increasingly prevalent chronic condition that is difficult for physicians to treat in their patients through diet and exercise alone. Gastrointestinal surgery is used by physicians to treat patients who are severely obese and cannot lose weight by traditional means or who suffer from serious obesity-related health problems. Generally, gastrointestinal surgery promotes weight loss by restricting food intake, and more specifically, by creating a narrow passage or “stoma” from the upper part of the stomach into the larger lower part of the stomach, which reduces the amount of food the stomach can hold and slows the passage of food through the stomach.

One of the more commonly used of these purely restrictive operations for obesity is adjustable gastric banding (AGB). In an exemplary AGB procedure, a hollow band (i.e., a gastric band) made of silicone elastomer is placed around the stomach near its upper end, creating a small pouch and a narrow passage (i.e., a stoma) into the rest of the stomach. The gastric band is then inflated with a saline solution by using a non-coring needle and syringe to access a small port that is placed underneath the skin. To control the size of the stoma, the gastric band can be tightened or loosened over time by the physician or another technician extracorporeally by increasing or decreasing the amount of saline solution in the gastric band via the access port to change the size of the stoma.

For example, a preferred gastric band for use is sold under the name LAP-BAND® Adjustable Gastric Banding System by Allergan, Inc. of Irvine, Calif., and is designed to be placed laparoscopically (via small incisions in the abdomen, usually 0.5-1.5 centimeters in length). An inflatable gastric band is placed around a top portion of a patient's stomach, creating a small pouch that limits or reduces food consumption. The LAP-BAND® AGB System is adjustable, which means that the inflatable gastric band can be tightened or loosened to help the patient achieve a level of satiety while maintaining a healthy diet, thereby supporting a patient's long-term weight loss success. Other possible gastric bands are adjustable electromechanically without hydraulics, and still others may have a fixed-size with no adjustment.

However, gastric bands might not be an appropriate solution for all patients. Accordingly, other forms of obesity prevention devices have been developed. For example, the ORBERA® System by Allergan, Inc. of Irvine, Calif. has been designed as a non-surgical and non-pharmaceutical weight loss system designed to lay the foundations for long-term, sustained weight loss.

The ORBERA® System is a smooth, spherical, intragastric balloon inserted endoscopically and filled with saline to assist with weight loss, by partially filling the stomach and inducing satiety. The ORBERA® System balloon functions to increase the likelihood of long-term maintenance of weight loss.

While the ORBERA® System is certainly a viable alternative to the gastric band, the ORBERA® System might not be optimized for patients who may benefit from an intragastric device that lasts inside the patient's stomach for an even longer period of time. In addition, the effectiveness of currently available methods of fighting obesity may be further enhanced by designing intragastric devices with even more advantageous geometry to promote weight loss.

Accordingly, the devices, methods and systems described herein are directed to improvements thereof.

### SUMMARY

The present application provides intragastric devices for the treatment of obesity. The intragastric devices advantageously act as volume-occupying devices that are able to survive implantation in a patient's stomach for a year or longer. The intragastric devices described herein may be implanted transorally, through the esophagus and into the patient's stomach without surgery or using only a minimally invasive surgical procedure. At the conclusion of treatment, the intragastric device may be retrieved gastroendoscopically. The intragastric device improves the overall efficacy of transoral obesity reducing devices by achieving a substantial reduction in device weight and may include an identical or improved space-occupying volume when compared to existing devices (e.g., the Orbera® System).

In one embodiment, an intragastric device may have a flat profile and configured to planarize the stomach by forming a wall-like divider in the stomach.

In another embodiment, an intragastric device may have a plurality of round protrusions or “bumps.”

In another embodiment, an intragastric device may have a plurality of legs, each of the plurality of legs having an enlarged end portion.

In another embodiment, an intragastric device may be substantially spherical and may include a plurality of protrusions spaced out on an outer surface of the intragastric device.

In another embodiment, an intragastric device may be substantially spherical and may include a plurality of spine-like or quill-like protrusions spaced out on an outer surface of the intragastric device.

In another embodiment, an intragastric device may be substantially spherical and may include a plurality of dimple-like recesses spaced out on an outer surface of the intragastric device.

For each of the embodiments described above, the intragastric device may be further configured to stimulate an inner stomach wall and/or temporarily block the pylorus to slow gastric emptying and/or be rotationally variant to encourage different stimulation points on the inner wall of the stomach, thereby limiting the ability of the stomach to adapt over long term implantation.

### BRIEF DESCRIPTION OF THE DRAWINGS

Features and advantages of the present invention will become appreciated as the same become better understood with reference to the specification, claims, and appended drawings wherein:

FIG. 1A is a perspective view of an intragastric device in accordance with one or more embodiments described herein.

FIG. 1B is a top view of the intragastric device of FIG. 1A in accordance with one or more embodiments described herein.

FIG. 1C is a side view along a first axis of the intragastric device of FIG. 1A in accordance with one or more embodiments described herein.

FIG. 1D is a side view along a second axis of the intragastric device of FIG. 1A in accordance with one or more embodiments described herein.

FIG. 2 is a perspective view of an intragastric device in accordance with one or more embodiments described herein.

FIG. 3 is a perspective view of an intragastric device in accordance with one or more embodiments described herein.

FIG. 4 is a perspective view of an intragastric device in accordance with one or more embodiments described herein.

FIG. 5 is a perspective view of an intragastric device in accordance with one or more embodiments described herein.

FIG. 6A is a perspective view of an intragastric device in accordance with one or more embodiments described herein.

FIG. 6B is a close up view of the intragastric device of FIG. 6A in accordance with one or more embodiments described herein.

### DETAILED DESCRIPTION

A number of different gastric devices are available today. However, the present disclosure offers improvements on the existing devices as described in greater detail below.

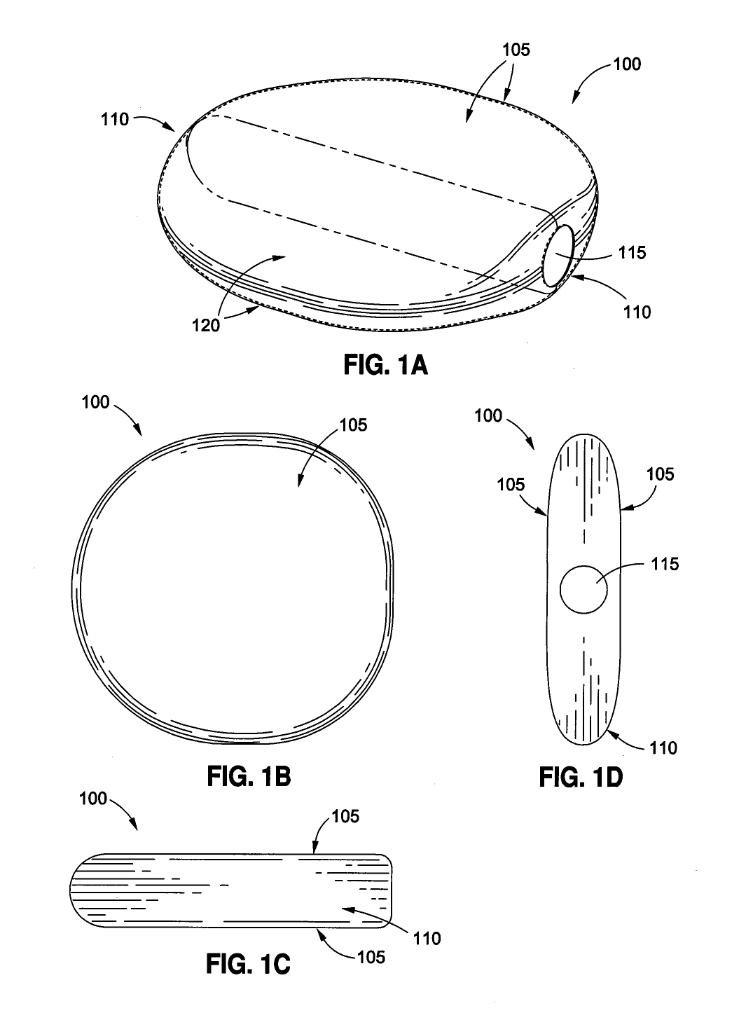

As seen in FIG. 1A, an intragastric device 100 may have a flat profile and may be configured to planarize the stomach by forming a wall-like divider in the stomach. More particularly, the geometry of the intragastric device 100 may be altered to stretch the stomach to a flatter geometry, which causes the volume capacity of the stomach to be substantially reduced.

The intragastric device 100 of FIG. 1A may include top and bottom surfaces 105, and an intermediate surface 110 substantially extending around the circumference of intragastric device 100. As shown, in FIG. 1A, the top and bottom surfaces 105 may comprise a plurality of joined panels 120 attached to each other and also attached to a circular panel 115 on an end substantially perpendicular to the top and bottom surfaces 105. As shown, the intragastric device 100 may be constructed of rubber, flourosilicones, flouroelastomers, thermoplastic elastomers or any combinations thereof. In one embodiment, the intragastric device 100 may be filled with air or a liquid such as silicon. The material(s) utilized may allow for a thinner wall thickness and have a lower coefficient of friction. Thinner walls and the lower coefficient of friction allows improved natural passage of the intragastric device 100 through the gastrointestinal tract should the intragastric device 100 deflate for any reason inside the patient's stomach.

FIG. 1B illustrates a top view of the intragastric device 100 of FIG. 1A. As illustrated, the intragastric device 100 is substantially circular. However, a different geometry may be implemented. For example, an intragastric device incorporating an oval or ellipse-shaped top or bottom surface is possible. While not shown, the top and bottom surfaces 105 may further include stimulation features such as rounded bumps or protrusions, quill-like extensions, dimples or recesses, and the like. These features, upon contact with the inner stomach wall of the patient may trigger hormone release or otherwise aid the patient in feeling full.

FIG. 1C illustrates a side view of the intragastric device 100 of FIG. 1A along one axis of the intragastric device 100. As shown, the thickness of the intermediate surface 110 substantially extending around the circumference of the intragastric device 100 is even. However, alternative configurations, including uneven thicknesses of the intermediate surface 100, may be possible.

FIG. 1D illustrates a side view of the intragastric device 100 of FIG. 1A along an axis of the intragastric device 100. Here, the circular panel 115 is shown to not extend to the top or bottom surface 105. In one embodiment, the circular panel 115 may include a port (not shown) for the inflation or deflation of the intragastric device 100.

The size of the intragastric device 100 may be configured such that the entire intragastric device 100 may be insertable transorally through the esophagus and into the stomach without invasive surgery. In one embodiment, the intragastric device 100 may be inserted into the patient's stomach using a standard grabber. In another embodiment, the intragastric device 100 may have a diameter of about 2-6 inches and may have a thickness of about 0.5-3 inches.

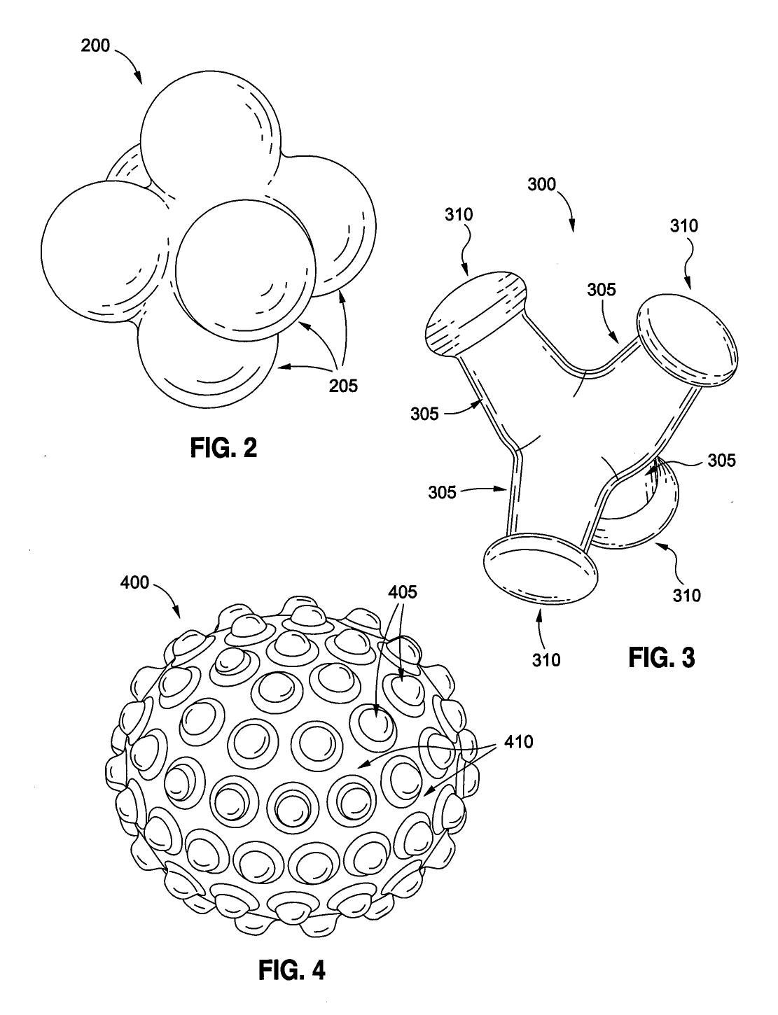

FIG. 2 illustrates another embodiment of the intragastric device 200. As shown, the intragastric device 200 has a plurality of protrusions 205 or “bumps” formed outwards from a center region of the intragastric device 200. While the intragastric device 200 may be sized to fit comfortably inside the patient's stomach, each of the plurality of protrusions 205 may be sized such that it blocks the patient's pylorus temporarily when the protrusion 205 comes into contact with the pylorus, thereby slowing gastric emptying and allowing the patient to feel full for a longer period of time without the protrusion 205 getting stuck or wedged into the pylorus. In addition, the configuration of the intragastric device 200 may produce variations in how the intragastric device 200 sits or rotates inside the patient's stomach. Normal stomach contractions may cause the intragastric device 200 to move around or rotate about the stomach, and due to the device's configuration, different points on the inner stomach walls may be stimulated, thereby limiting the stomach's ability to adapt over a long period of time.

In one embodiment, the protrusions 205 may be placed in an asymmetrical pattern to further limit the ability of the stomach to adapt over a long period of time.

The intragastric device 200 may be constructed of rubbers, fluorosilicones, flouroelastomers, thermoplastic elastomers or any combination thereof to improve the durability of the intragastric device 200 inside the patient's stomach. However, the intragastric device 200 may be constructed of a continuous, thin, depressable wall and be hollow inside (filled with air) to keep the intragastric device 200 light. Alternatively, the intragastric device 200 may be filled with a liquid gel such as silicon. The material(s) utilized to construct the intragastric device 200 may allow for a thinner wall thickness and have a lower coefficient of friction. Thinner walls and the lower coefficient of friction allow improved natural passage of the intragastric device 200 through the gastrointestinal tract should the intragastric device 200 deflate for any reason inside the patient's stomach.

While not shown, the outer surface of the intragastric device 200 may further include additional stimulation features such as even smaller rounded bumps or protrusions formed on the protrusions 205 (e.g., 10-15 mini-protrusions on each of the six protrusions shown in FIG. 2, equally spread apart and having a substantially similar shape, but with a smaller diameter as compared to the protrusion 205), quill-like extensions, dimples or recesses, and the like. These features, upon contact with the inner stomach wall of the patient may further trigger hormone release or otherwise aid the patient in feeling full.

FIG. 3 illustrates another embodiment of the intragastric device 300. As shown, the intragastric device 300 has four “legs”. The configuration of the four legs may be asymmetrical as shown. If divided into “the top two legs” and “the bottom two legs”, the “pairs of legs” appear joined at the center and “twisted 90 degrees” to form the configuration as shown. However, this is merely one example of any of a plurality of configurations for any of a plurality of leg numbers. For example, additional legs may be attached or removed, and/or the configuration may be altered. In addition, each leg portion 305 may be joined to one end 310. The ends 310, as shown, caps the end of the leg portion 305 and has a diameter substantially thicker than the diameter of the leg portion 305. Accordingly, the ends 310 may be sized such that it blocks the patient's pylorus temporarily when the ends 310 comes into contact with the pylorus, and thereby slowing gastric emptying and allowing the patient to feel full for a longer period of time without the ends 310 getting stuck or wedged into the pylorus. In addition, the configuration of the intragastric device 300 may produce variations in how the intragastric device 300 sits or rotates inside the patient's stomach. Normal stomach contractions may cause the intragastric device 300 to move around or rotate about the stomach, and due to the device's configuration, different points on the inner stomach walls may be stimulated, thereby limiting the ability of the stomach to adapt over a long period of time.

The intragastric device 300 may be constructed of rubbers, fluorosilicones, flouroelastomers, thermoplastic elastomers or any combination thereof to improve the durability of the intragastric device 300 inside the patient's stomach. However, the intragastric device 300 may be constructed of a continuous, thin, depressable wall and be hollow inside (filled with air) to keep the intragastric device 300 light. Alternatively, the intragastric device 300 may be filled with a liquid gel such as silicon. The materials utilized may allow for a thinner wall thickness and have a lower coefficient of friction. Thinner walls and the lower coefficient of friction allows improved natural passage of the intragastric device 300 through the gastrointestinal tract should the intragastric device 300 deflate for any reason inside the patient's stomach.

While not shown, the outer surface of the intragastric device 300 may further include additional stimulation features such as even small rounded bumps or protrusions formed on the ends 310 (e.g., 10-15 mini-protrusions on each of the four ends shown in FIG. 3, equally spread apart and having a substantially similar shape, but with a much smaller diameter as compared to the ends 310), quill-like extensions, dimples or recesses, and the like. These features, upon contact with the inner stomach wall of the patient may further trigger hormone release or otherwise aid the patient in feeling full.

FIG. 4 illustrates another embodiment of the intragastric device 400. As shown, the intragastric device 400 is a substantially spherical object with protrusions or bumps 405 extending outward from the surface of the intragastric device 400. As shown, a plurality of protrusions 405 may be equally spaced apart on the outer surface and interspersed with flat portions 410. In one embodiment, the protrusions 405 do not contact each other. In another embodiment, the protrusions 405 may be of equal heights and diameters. However, the protrusions 405 may be configured to contact each other (and thereby creating space and allowing for more protrusions 405 to be added to the surface). In another embodiment, the protrusions 405 may be configured to have different heights and/or diameters. For example, having protrusions with different heights and/or diameters may be advantageous for preventing the stomach from adjusting to the protrusions 405.

In another embodiment, the size of the intragastric device 400 may be altered. For example, in a uni-intragastric device system, one intragastric device 400 may be implanted into the patient's stomach, and the single intragastric device may be sized accordingly to fit comfortably inside the patient's stomach. However, it is also possible to employ multiple, smaller devices, such as 2 or 3 objects similar to the intragastric device 400. Under this multi-intragastric device system, each intragastric device 400 may be sized such that it blocks the patient's pylorus temporarily when the intragastric device 400 comes into contact with the pylorus, and thereby slowing gastric emptying and allowing the patient to feel full for a longer period of time (and also to prevent intestinal blockage). In addition, the configuration of the intragastric device 400 may produce variations in how the intragastric device 400 sits or rotates inside the patient's stomach. Normal stomach contractions may cause the intragastric device 400 to move around or rotate about the stomach, and due to the device's configuration, different points on the inner stomach walls may be stimulated, thereby limiting the ability of the stomach to adapt over a long period of time.

The intragastric device 400 may be constructed of rubbers, fluorosilicones, flouroelastomers, thermoplastic elastomers or any combination thereof to improve the durability of the intragastric device 400 inside the patient's stomach. However, the intragastric device 400 may be constructed of a continuous, thin, depressable wall and be hollow inside (filled with air) to keep the intragastric device 400 light. Alternatively, the intragastric device 400 may be filled with a liquid gel such as silicon. Regardless, the materials utilized may allow for a thinner wall thickness and have a lower coefficient of friction. Thinner walls and the lower coefficient of friction allows improved natural passage of the intragastric device 400 through the gastrointestinal tract should the intragastric device 400 deflate for any reason inside the patient's stomach.

While not shown, the outer surface of the intragastric device 400 may further include additional stimulation features such as even small rounded bumps or protrusions formed on the protrusions 405 (e.g., 10-15 mini-protrusions on each of the protrusions 405 of FIG. 4, equally spread apart and having a substantially similar shape, but with a much smaller diameter as compared to the protrusions 405), quill-like extensions, dimples or recesses, and the like. These features, upon contact with the inner stomach wall of the patient may further trigger hormone release or otherwise aid the patient in feeling full.

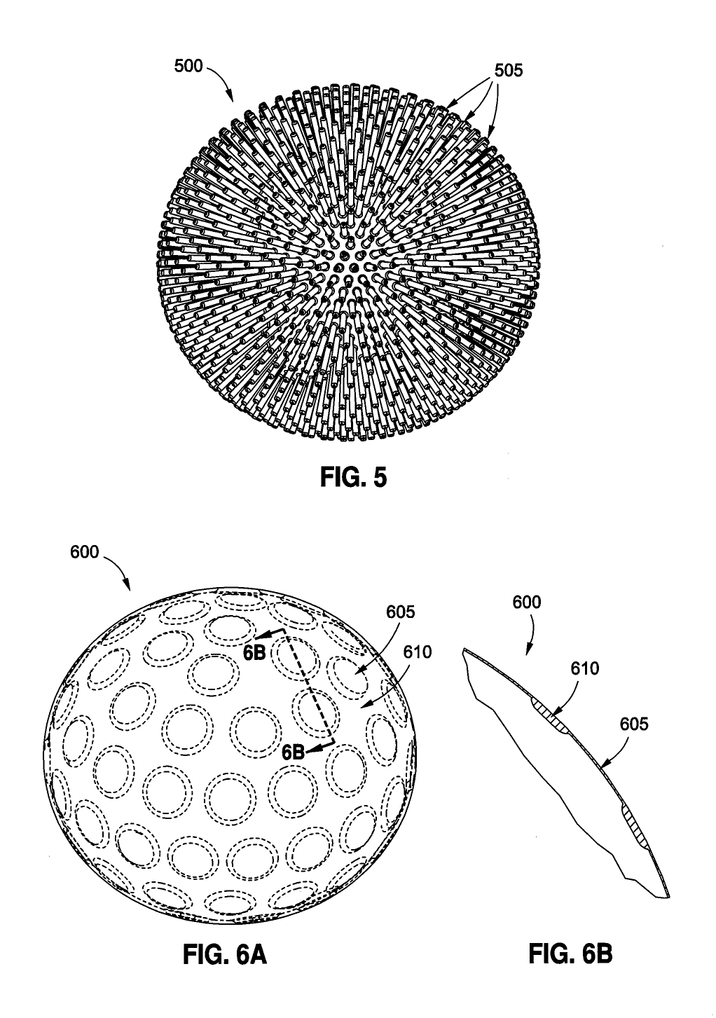

FIG. 5 illustrates another embodiment of the intragastric device 500. As shown, the intragastric device 500 is a substantially spherical object with long, quill-like extensions 505 extending outward from the outer surface of a central region of the intragastric device 500. As shown, a plurality of extensions 505 may be equally spaced apart. In one embodiment, the extensions 505 do not contact each other. In another embodiment, the extensions 505 may be of equal heights and diameters. However, the extensions 505 may be configured to contact each other (and thereby creating space and allowing for more extensions 505 to be added to the surface). In another embodiment, the extensions 505 may be configured to have different heights and/or diameters. For example, having protrusions with different heights and/or diameters may be advantageous for preventing the stomach from adjusting to the extensions 505. In one embodiment, the extensions may be extremely flexible and may bend when a pressure is exerted on the extensions 505 from the inner stomach wall of the patient. Alternatively, the extensions 505 may be stiffer and might not bend as much when a pressure is exerted on the extensions 505 from the inner stomach wall of the patient. In another embodiment, some of the extensions 505 may have a first flexibility while some of the extensions may have a second flexibility. Alternatively, the extensions 505 may be uniformly flexible. In other words, any flexibility of the extensions may be utilized with the intragastric device 500.

In another embodiment, the size of the intragastric device 500 may be altered. For example, in a uni-intragastric device system, one intragastric device 500 may be implanted into the patient's stomach, and the single intragastric device may be sized accordingly to fit comfortably inside the patient's stomach. However, it is also possible to employ multiple, smaller devices, such as 2 or 3 objects similar to the intragastric device 500. Under this multi-intragastric device system, each intragastric device 500 may be sized such that it blocks the patient's pylorus temporarily when the intragastric device 500 comes into contact with the pylorus, and thereby slowing gastric emptying and allowing the patient to feel full for a longer period of time (and also to prevent intestinal blockage). In addition, the configuration of the intragastric device 500 may produce variations in how the intragastric device 500 sits or rotates inside the patient's stomach. Normal stomach contractions may cause the intragastric device 500 to move around or rotate about the stomach, and due to the device's configuration, different points on the inner stomach walls may be stimulated, thereby limiting the ability of the stomach to adapt over a long period of time. In another embodiment of the multi-intragastric device system, different intragastric devices may be implanted into the same patient's stomach at the same time. For example, the intragastric device of FIG. 4 and the intragastric device of FIG. 5 may both be implanted in the patient and may simultaneously work together. One benefit of this approach may be that the stomach will have an even more difficult time adjusting to the intragastric devices 400 and 500 since they are completely different from one another, thereby improving the efficacy of the system.

Referring back to FIG. 5, the intragastric device 500 may be constructed of rubbers, fluorosilicones, flouroelastomers, thermoplastic elastomers or any combination thereof to improve the durability of the intragastric device 500 inside the patient's stomach. However, the intragastric device 500 may be constructed of a continuous, thin, depressable wall and be hollow inside (filled with air) to keep the intragastric device 500 light. Alternatively, the intragastric device 500 may be filled with a liquid gel such as silicon. Regardless, the materials utilized may allow for a thinner wall thickness and have a lower coefficient of friction. Thinner walls and the lower coefficient of friction allows improved natural passage of the intragastric device 500 through the gastrointestinal tract should the intragastric device 500 deflate for any reason inside the patient's stomach.

FIG. 6A illustrates another embodiment of the intragastric device 600. As shown, the intragastric device 600 is a substantially spherical object with recesses or dimples 605 extending inward from the surface of the intragastric device 600. In one embodiment, the intragastric device 600 may be considered to have a surface comprised of recesses 605 and flat portions 610. As shown, a plurality of recesses 605 may be equally spaced apart on the outer surface. As shown, recesses 605 do not contact each other, and may be of equal heights and diameters. In addition to being depressed, the recesses 605 may employ a thinner wall. For example, if the flat portions 610 have a wall thickness of 20 millimeters, the recesses 605 may have a wall thickness of 10 millimeters. With a thinner wall, the recesses 605 may be more susceptible to larger strains.

The intragastric device 600 is effectively triggered in the patient's stomach by stomach contractions. These stomach contractions increase the pressure in the intragastric device 600. FIG. 6B illustrates a close up view of the recesses 605 and the flat portions 610. If the recess 605 is not in contact with the stomach wall or some outside retaining force, the recess 605 with the thinner walls will deform until the recess 605 comes into contact with the stomach wall or comes under the influence of some other outside force. The recess 605 will also stop deforming when no contact is made when the modulus of the material forming the recess is such that the stress in the material is balanced with the internal pressure of the intragastric device 600.

Now, if the recess 605 is in contact with the stomach wall, the pressure exerted on the recess 605 may cause the intragastric device 600 to exert a disproportionately larger force on the stomach wall (as compared to the immediate surround area, e.g., the non-recessed, flat portions 610).

In another embodiment, the size of the intragastric device 600 may be altered. For example, in a uni-intragastric device system, one intragastric device 600 may be implanted into the patient's stomach, and the single intragastric device may be sized accordingly to fit comfortably inside the patient's stomach. However, it is also possible to employ multiple, smaller devices, such as 2 or 3 objects similar to the intragastric device 600. Under this multi-intragastric device system, each intragastric device 600 may be sized such that it blocks the patient's pylorus temporarily when the intragastric device 600 comes into contact with the pylorus, and thereby slowing gastric emptying and allowing the patient to feel full for a longer period of time (and also to prevent intestinal blockage). In addition, the configuration of the intragastric device 600 may produce variations in how the intragastric device 600 sits or rotates inside the patient's stomach. Normal stomach contractions may cause the intragastric device 600 to move around or rotate about the stomach, and due to the device's configuration, different points on the inner stomach walls may be stimulated, limiting the ability of the stomach to adapt over a long period of time.

The intragastric device 600 may be constructed of rubbers, fluorosilicones, flouroelastomers, thermoplastic elastomers or any combination thereof to improve the durability of the intragastric device 600 inside the patient's stomach. In one embodiment, the flat portions 610 and the recesses 605 may be constructed of different materials. For example, the flat portions 610 may be made of one material with one mechanical property (e.g., a rubber) while the recesses 605 may be constructed of a different material with a different mechanical property (e.g., a thermoplastic elastomer).

Alternatively, the intragastric device 600 may constructed of a continuous, thin, depressable wall of the same material, but of different thicknesses (e.g., the flat portions 610 may be thicker than the recesses 605). In one embodiment, the intragastric device 600 may be hollow inside (filled with air) to keep the intragastric device 600 light. Alternatively, the intragastric device 600 may be filled with a liquid gel such as silicon. The materials utilized may allow for a thinner wall thickness and have a lower coefficient of friction. Thinner walls and the lower coefficient of friction allows improved natural passage of the intragastric device 600 through the gastrointestinal tract should the intragastric device 600 deflate for any reason inside the patient's stomach.

While not shown, the outer surface of the intragastric device 600 may further include additional stimulation features such as small rounded bumps or protrusions formed on the flat portions 610, quill-like extensions, and the like. These features, upon contact with the inner stomach wall of the patient may further trigger hormone release or otherwise aid the patient in feeling full.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, references may have been made to patents and printed publications in this specification. Each of the above-cited references and printed publications are individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1.-20. (canceled)

21. An intragastric device for occupying space in a patient's stomach and for treating obesity, the intragastric device comprising:
a plurality of round protrusions attached to one another and projecting outward from a center of the intragastric device, the protrusions configured to permit rotation of the intragastric device as stomach muscles contracts, wherein each protrusion can substantially block the entrance of the pylorus to the stomach, without residing within the pylorus, thereby impeding flow of food into the stomach, the device capable of remaining in the stomach and functioning therein for at least one year to treat obesity.

22. The intragastric device of claim 21, wherein the protrusions are relatively large partial-spheres.

23. The intragastric device of claim 21, wherein the protrusions are each sized to temporarily block the pylorus of the patient when the protrusion contacts the pylorus but are too large to fit within the pyloris opening.

24. The intragastric device of claim 21, wherein there are a total of six protrusions.

25. The intragastric device of claim 21, wherein the protrusions are placed in an asymmetrical pattern around the intragastric device.

26. The intragastric device of claim 21, wherein the intragastric device is hollow and filled with air.

27. The intragastric device of claim 21, wherein the intragastric device is hollow and filled with a liquid gel.

28. The intragastric device of claim 21, wherein an exterior surface of each protrusion is not smooth but has a plurality of mini surface stimulation features thereon.

29. An intragastric device for occupying space in a patient's stomach and for treating obesity, the intragastric device comprising:
a plurality of legs attached to one another and projecting outward from a center of the intragastric device, the plurality of legs configured such that the intragastric device is rotationally variant depending on natural contractions of the patient's stomach, wherein each leg terminates in a leg end larger than the rest of the leg and sized to temporarily block the pylorus of the patient when the leg end contacts the pylorus but being too large to fit within the pyloris opening, the device further configured to be functional inside the patient's stomach for a year or more.

30. The intragastric device of claim 29, wherein there are a total of four legs.

31. The intragastric device of claim 29, wherein the legs are placed in an asymmetrical pattern around the intragastric device.

32. The intragastric device of claim 29, wherein the intragastric device is hollow and filled with air.

33. The intragastric device of claim 29, wherein the intragastric device is hollow and filled with a liquid gel.

34. The intragastric device of claim 29, wherein an exterior surface of each leg end is not smooth but has a plurality of mini surface stimulation features thereon.

35. An intragastric device for occupying space in a patient's stomach, the intragastric device comprising:
a hollow body having an interior chamber and an exterior surface defined by a wall of the hollow body, wherein the exterior surface is not smooth but has a plurality of mini surface stimulation features thereon, the body further configured to be functional inside the patient's stomach for a year or more.

36. The intragastric device of claim 35, wherein the mini surface stimulation features comprises rounded protruding portions alternating with non-protruding portions.

37. The intragastric device of claim 36, wherein at least 3 of the plurality of protrusions have different heights and diameters than other of the protrusions.

38. The intragastric device of claim 35, wherein the mini surface stimulation features comprises a plurality of quill-like extensions.

39. The intragastric device of claim 38, wherein at least two of the plurality of the quill-like extensions have different flexibilities than other of the quill-like extensions.

40. The intragastric device of claim 38, wherein each of the quill-like extensions has a diameter of less than 5 mm.

41. The intragastric device of claim 35, wherein the mini surface stimulation features comprise a plurality of dimpled recesses having a thinner wall thickness than surrounding flat portions of the hollow body wall.

42. The intragastric device of claim 41, wherein the flat portions have a wall thickness of about half the wall thickness of the dimpled recesses.
